(19) United States
(12) Patent Application Publication
(10) Pub. No.: US 2020/0070146 A1
(43) Pub. Date: Mar. 5, 2020

(54) Systems And Methods For Simultaneous Detection And Identification Of Microorganisms Within A Fluid Sample

(71) Applicant: BacterioScan, Inc., St. Louis, MO (US)

(72) Inventor: Andrew Tomaras, Wildwood, MO (US)

(21) Appl. No.: 16/546,805

(22) Filed: Aug. 21, 2019

Related U.S. Application Data
(60) Provisional application No. 62/725,165, filed on Aug. 30, 2018.

(51) Int. Cl. B01L 3/00; G01N 21/01; G01N 21/51; G01N 1/10; G01N 33/569

(52) U.S. Cl. CPC B01L 3/5025; G01N 21/01; G01N 21/51; G01N 1/10; G01N 33/56911; B01L 2300/08; B01L 2300/0864; G01N 2001/1006; B01L 2200/0652; B01L 2300/12; G01N 2201/0231

(57) ABSTRACT

An optical measurement instrument is an integrated instrument that includes an optical cavity with a light source, a sample cuvette, and an optical sensor. The light source and sensor are on a bench that is on a translational or rotational mechanical platform such that optical beam can be moved to multiple sample containers. Each sample containers holds a distinct microorganism-attracting substance and a portion of a fluid sample containing an unknown microorganism. Each distinct microorganism-attracting substance is configured to bind with a single type of microorganism. The unknown microorganism in the fluid sample binds with the distinct microorganism-attracting substance in a single sample container. The instrument incubates the microorganism in the single sample container and detects the presence of the microorganism in the single sample container to thereby simultaneously identify the unknown microorganism.

### CROSS-REFERENCE TO RELATED APPLICATIONS

This application claims priority to and the benefit of U.S. Provisional Patent Application No. 62/725,165, filed Aug. 30, 2018, and titled “Systems And Methods For Simultaneous Detection And Identification Of Microorganisms Within A Fluid Sample,” which is incorporated herein by reference in its entirety.

### COPYRIGHT

A portion of the disclosure of this patent document may contain material which is subject to copyright protection. The copyright owner has no objection to the facsimile reproduction by anyone of the patent disclosure, as it appears in the Patent and Trademark Office patent files or records, but otherwise reserves all copyright rights whatsoever.

### FIELD OF THE INVENTION

The present invention relates generally to the field of measurements of biological liquid samples. Specifically, the present invention relates to systems and method for simultaneously detecting and identifying microorganisms within the liquid sample.

### BACKGROUND OF THE INVENTION

Many applications in the field of analytical research and clinical testing utilize methods for analyzing liquid samples. Among those methods are optical measurements that measure absorbance, turbidity, fluorescence/luminescence, and optical scattering measurements. Optical laser scattering is one of the most sensitive methods, but its implementation can be very challenging, especially when analyzing biological samples in which suspended particles are relatively transparent in the medium. In addition, preparing and measuring biological samples can be costly and time-consuming due to the need to ensure a sufficient volume of the sample or of any unknown microorganisms within the sample. Accordingly, there is a need for improved devices, systems, and methods that can quickly and simultaneously detect and identify a microorganism present in a fluid sample.

### SUMMARY OF THE INVENTION

According to aspects of the present disclosure, a specimen collection device comprises an inlet and a plurality of fluid containers in fluid communication with the inlet. Each of the plurality of fluid containers includes a distinct microorganism-attracting substance disposed therein. The distinct microorganism-attracting substance of each of the plurality of fluid containers is configured to attract a respective one of a plurality of types of microorganisms such that each of the plurality of fluid containers is associated with the respective one of the plurality of types of microorganisms.

According to other aspects of the present disclosure, a specimen collection device comprises an inlet, a first fluid container in fluid communication with the inlet, and a second fluid container. The first fluid container includes a first microorganism-attracting substance disposed therein configured to attract a first type of microorganism. The second fluid container is in fluid communication with the inlet and includes a second microorganism-attracting substance disposed therein configured to attract a second type of microorganism.

According to additional aspects of the present disclosure, an optical measuring instrument for detecting and identifying a microorganism in a fluid sample comprises a housing with a substantially light-tight enclosure and a plurality of fluid containers. Each of the fluid containers holds a portion of the fluid sample and a distinct microorganism-attracting substance. The distinct microorganism-attracting substance of each of the plurality of fluid containers is configured to attract a respective one of a plurality of types of microorganisms such that each of the plurality of fluid containers is associated with the respective one of the plurality of types of microorganisms. Each of the fluid containers has an input window and an output window. The instrument also includes a light source within the housing to provide an input beam for transmission into the input windows of the fluid containers and though the corresponding portions of the fluid sample. The input beam creates a forward-scatter signal for each of the fluid containers. Each of the forward-scatter signals is associated with the presence and concentration of the respective one of the plurality of types of microorganisms associated with each of the plurality of fluid containers. The instrument further includes at least one sensor within the housing for detecting the forward-scatter signals exiting from the output windows, and a heating element within the housing to maintain the portions of the fluid sample at a desired temperature to encourage microorganism growth in the portions of the fluid sample over a period of time. At least one of the input beam and the fluid containers is movable relative to each other so that the input beam sequentially addresses each of the plurality of fluid samples.

According to still other aspects of the present disclosure, an optical measuring instrument for detecting and identifying a microorganism in a fluid sample comprises a plurality of cuvette assemblies having optical chambers for receiving a respective portion of the fluid sample. Each of the optical chambers includes (i) a distinct microorganism-attracting substance configured to attract a respective one of a plurality of types of microorganisms such that each of the plurality of optical chambers is associated with the respective one of the plurality of types of microorganisms, (ii) an entry window for allowing transmission of an input light beam through the respective portion of the fluid sample, and (iii) an exit window for transmitting an optical signal caused by the respective one of the plurality of types of microorganisms within the respective portion of the fluid sample. Each cuvette assembly has a first pair of registration structures associated therewith. The instrument includes a platform structure with multiple second pairs of registration structures for mating with the first pair of registration structures of the plurality of cuvette assemblies. The instrument includes a light source producing the input light beam and a sensor for receiving the optical signal caused by the bacteria.

According to still additional aspects of the present disclosure, an optical measuring instrument for detecting and identifying a microorganism in a fluid sample comprises a plurality of cuvette assemblies having optical chambers for receiving a respective portion of the fluid sample. Each of the optical chambers includes (i) a distinct microorganism-attracting substance configured to attract a respective one of a plurality of types of microorganisms such that each of the plurality of optical chambers is associated with the respective one of the plurality of types of microorganisms, (ii) an entry window for allowing transmission of an input light beam through the respective portion of the fluid sample, and (iii) an exit window for transmitting an optical signal caused by the respective one of the plurality of types of microorganisms within the respective portion of the fluid sample. The instrument includes a heating system that permits a controlled incubation of the portions of the fluid sample, a light source for producing the input light beam, a sensor for receiving the optical signal. The light source is periodically operational during the controlled incubation so as to allow the sensor to receive a series of optical signals that are used to detect and identify at least one of the plurality of types of microorganisms, and to determine a concentration of the at least one of the plurality of types of microorganisms.

According to other aspects of the present disclosure, a method of detecting and (i) identifying a microorganism in a fluid sample comprises placing a portion of the fluid sample in each of a plurality of fluid containers, each fluid container including a distinct microorganism-attracting substance disposed therein, the distinct microorganism-attracting substance of each of the plurality of fluid containers being configured to attract a respective one of a plurality of types of microorganisms such that each of the plurality of fluid containers is associated with the respective one of the plurality of types of microorganisms, each fluid container having a first window for receiving an input beam and a second window for transmitting a forward-scatter signal caused by the input beam; (ii) inserting each of the fluid containers into an optical measuring instrument; (iii) incubating the portions of the fluid sample in the optical measuring instrument; (iv) within the optical measuring instrument, sequentially passing the input beam through each portion of the fluid sample and measuring a first forward-scatter signal for each portion of the fluid sample; (v) continuing to incubate the portions of the fluid sample within the optical measuring instrument for a period of time; and (vi) after the period of time, sequentially passing the input beam through each portion of the fluid sample and measuring a second forward-scatter signal for each portion of the fluid sample, a difference between the first forward-scatter signal and the second forward-scatter signal for each portion of the fluid sample being indicative of a presence and an identity of at least one of the plurality of types of microorganisms within the portion of the fluid sample, and a change in a concentration of the at least one of the plurality of types of microorganisms within the portion of the fluid sample.

According to additional aspects of the present disclosure, a method of detecting and identifying a microorganism in a fluid sample comprises within the optical measuring instrument, incubating the fluid sample while a portion of the fluid sample is within a corresponding one of a plurality of cuvette chambers, each cuvette chamber having (i) a distinct microorganism-attracting substance disposed therein, the distinct microorganism-attracting substance of each of the plurality of cuvette chambers being configured to attract a respective one of a plurality of types of microorganisms such that each of the plurality of cuvette chambers is associated with the respective one of the plurality of types of microorganisms, (ii) a first window for receiving an input beam, and (iii) a second window for transmitting a forward-scatter signal caused by the input beam. The method further includes, during the incubating, repeatedly transmitting the input beam through each portion of the fluid sample and measuring a series of forward-scatter signals for each portion of the fluid sample. The method also includes determining that at least one portion of the fluid sample includes a concentration of the respective one of the plurality of types of microorganisms in response to changes in the forward-scatter signals within the series of forward-scatter signals for the at least one portion of the fluid sample.

According to other aspects of the present disclosure, a method of detecting and identifying a microorganism in a fluid sample, the fluid sample containing the microorganism and at least one other substance comprises (i) placing a portion of the fluid sample in each of a plurality of fluid containers, each fluid container including a distinct microorganism-attracting substance disposed therein, the distinct microorganism-attracting substance of each of the plurality of fluid containers being configured to attract a respective one of a plurality of types of microorganisms such that each of the plurality of fluid containers is associated with the respective one of the plurality of types of microorganisms; (ii) agitating each of the plurality of fluid containers such that the distinct microorganism-attracting substance disposed in a first one of the plurality of fluid containers binds with the microorganism in the fluid sample; (iii) removing the at least one other substance from each of the plurality of fluid containers and retaining the distinct microorganism-attracting substance in each of the plurality of fluid containers such that the microorganism in the fluid sample is retained only in the first one of the plurality of fluid containers; (iv) incubating each of the plurality of fluid containers; (v) passing an input beam through each of the fluid containers and measuring a first forward-scatter signal for each of the plurality of fluid containers; (vi) continuing to incubate the plurality of fluid containers for a period of time; and (vii) after the period of time, passing the input beam through each of the plurality of fluid containers and measuring a second forward-scatter signal for each of the plurality of fluid containers, a difference between the first scatter signal and the second scatter signal for the first one of the plurality of fluid containers indicating a presence of the microorganism in the first one of the plurality of fluid containers and an identity of the microorganism.

Additional aspects of the invention will be apparent to those of ordinary skill in the art in view of the detailed description of various embodiments, which is made with reference to the drawings, a brief description of which is provided below.

### BRIEF DESCRIPTION OF THE DRAWINGS

FIG. 1A illustrates an optical-measuring instrument that is capable of incubating fluid samples by having a controlled internal heating system.

FIG. 1B illustrates the cuvettes of FIG. 2 being placed and registered within the optical-measuring instrument of FIG. 1A.

FIG. 2 illustrates four multi-chamber cuvettes that receive fluid samples that are placed in the optical-measuring device of FIGS. 1A and 1B.

FIG. 3 illustrates a side schematic view of the optical-measuring instrument of FIGS. 1A and 1B.

FIG. 4 illustrates a top schematic view of the optical-measuring instrument of FIGS. 1A and 1B.

FIG. 5 illustrates a system control diagram for the optical-measuring instrument of FIGS. 1A and 1B.

FIG. 6 is an exploded view of one the multi-chamber cuvettes of FIG. 2 that is used with the optical-measuring device of FIGS. 1A and 1B.

FIG. 7 is a cross-sectional view through one chamber of the multi-chamber cuvette of FIG. 2 that is used with the optical-measuring device of FIGS. 1A and 1B.

FIG. 8 illustrates the cuvette assembly of FIGS. 2, 6 and 7 registered on a platform or tray (typically heated) that is movable from the open position in which the instrument's door is opened (FIG. 1B) for loading to the closed position in which the instrument's door is closed (FIG. 1A) for sample testing within the optical measurement instrument of FIGS. 1A and 1B

FIG. 9 illustrates an alternative optical-measuring instrument that is capable of incubating fluid samples in which the cuvettes form part of the light-tight closure of the optical-measuring instrument.

FIG. 10 is an isometric view of optical-measuring instrument with fixed optical elements and multiple cuvettes that are rotated on a rotatable platform into the light beam for measuring optical characteristics of multiple samples.

FIG. 11 illustrates a molecule of a microorganism-attracting substance including an affinity body and multiple ligands coupled to the affinity body.

FIG. 12 illustrates a flow diagram for detecting and identifying a microorganism in a fluid sample using one or more types of the microorganism-attracting substance of FIG. 11.

FIG. 13 illustrates a specimen collection device for use with one or more types of the microorganism-attracting substance of FIG. 11.

While the invention is susceptible to various modifications and alternative forms, specific embodiments will be shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

The drawings will herein be described in detail with the understanding that the present disclosure is to be considered as an exemplification of the principles of the invention and is not intended to limit the broad aspect of the invention to the embodiments illustrated. For purposes of the present detailed description, the singular includes the plural and vice versa (unless specifically disclaimed); the words “and” and “or” shall be both conjunctive and disjunctive; the word “all” means “any and all”; the word “any” means “any and all”; and the word “including” means “including without limitation.”

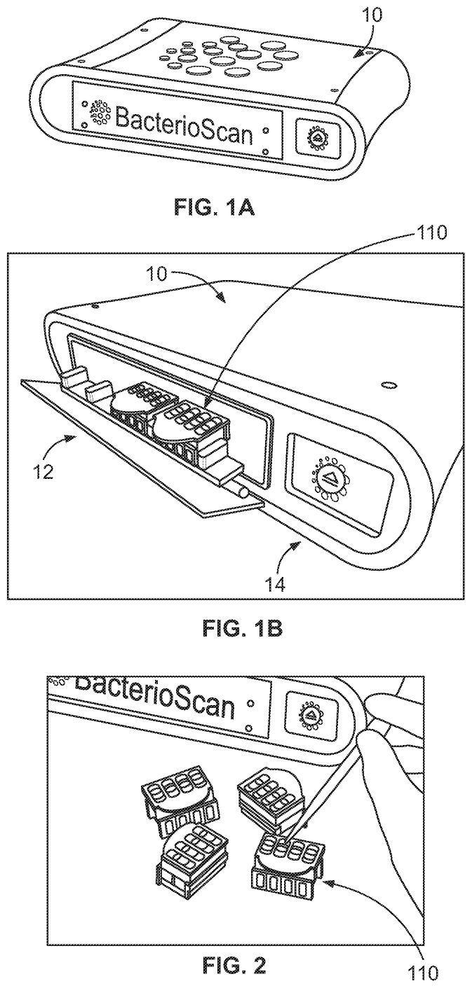

FIG. 1A illustrates an optical measuring device 10 (manufactured by the assignee of the present application as the BacterioScan 216R instrument) that can rapidly detect and quantify the concentration of bacteria in a fluid sample. As discussed in more detail below, the instrument 10 includes on-board incubation, such that reagents to enhance growth are not necessarily needed (although they can be used). The instrument 10 uses laser-scattering technology to quantify bacteria growth in fluid sample sizes as small as 1 milliliter. In particular, the instrument 10 transmits a laser beam through a fluid sample, and measures the scatter signal caused by the bacteria in the fluid sample, preferably through a forward-scattering measurement technique. The on-board incubation provides for fluid sample temperatures ranging from room temperature up to 42° C. (or higher). The instrument 10 permits for a range of optical measurement intervals over a period of time (e.g., 1-6 hours) to determine the growth and concentration of the bacteria within the liquid samples during incubation. The optical measuring instrument 10 can detect and count bacteria by various techniques that are generally described in U.S. Pat. Nos. 7,961,311 and 8,339,601, both of which are commonly owned and are herein incorporated by reference in their entireties.

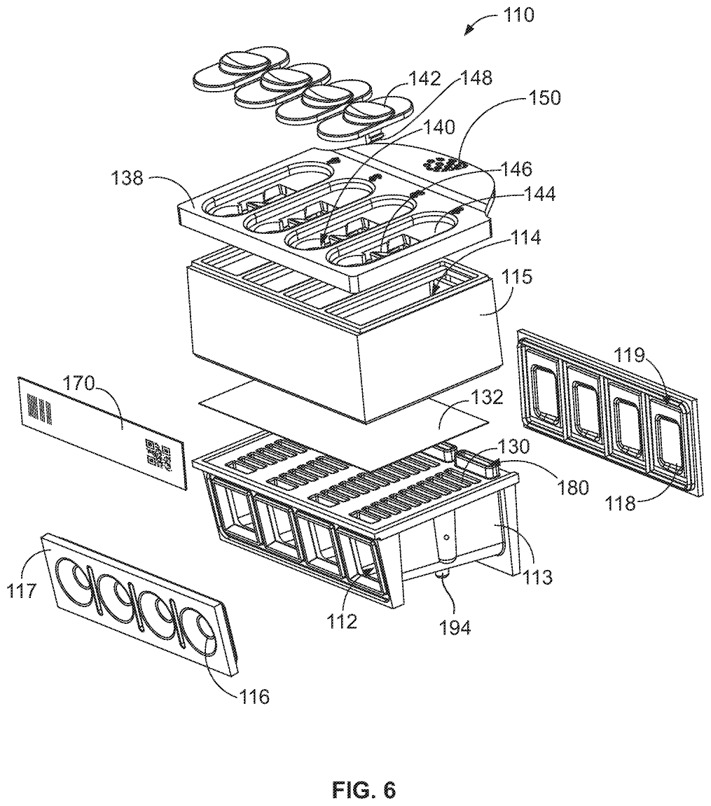

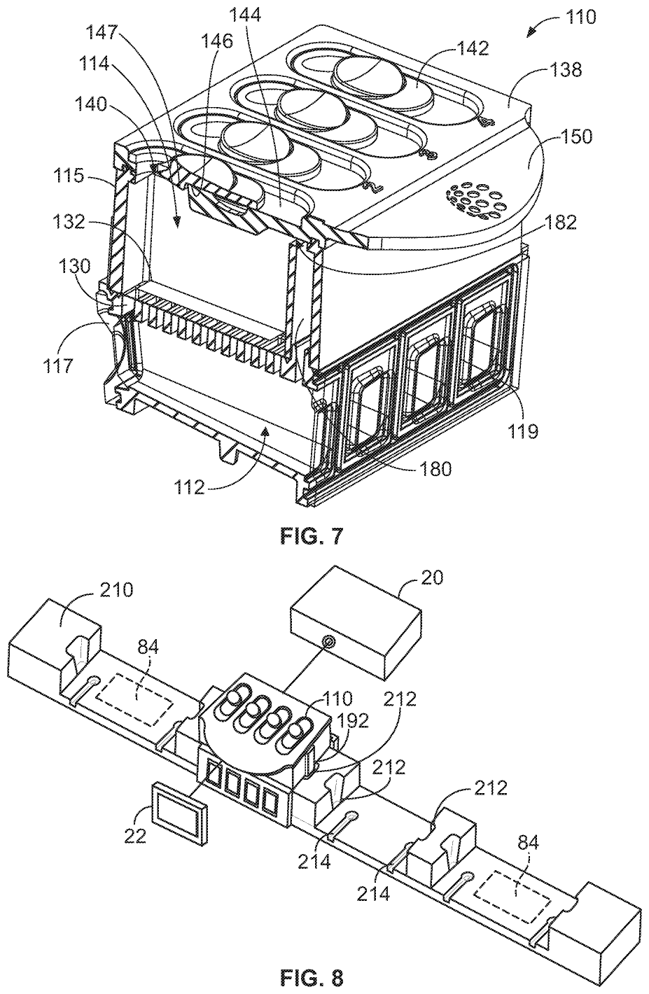

FIG. 1B illustrates cuvette assemblies 110 being inserted into the optical measurement instrument 10 of FIG. 1A. To do so, a front door 12 on the optical measurement instrument 10 is opened and the cuvette assemblies 110 are placed on a registration and orientation plate or platform 210 (See FIG. 8) such that the laser-input window and output-signal window of each cuvette (FIGS. 6-7) are substantially registered within the optical measurement instrument 10, permitting periodic optical measurements to be taken of each sample. As shown, the optical measurement instrument 10 may include up to four cuvettes 110, such that 16 different samples can be tested periodically through the optical measurement instrument 10.

The optical measuring instrument 10 includes a display device 14 that provides information regarding the tests and/or fluid samples. For example, the display device 14 may indicate the testing protocol being used for the samples (e.g., time and temperature) or provide the current temperature within the instrument 10. Preferably, the display device 14 also includes an associated touchscreen input (or a different set of input buttons can be provided) that allows a user to perform some of the basic functions of the instrument 10, such as a power on/off function, a door open/close function, a temperature increase/decrease function, etc.

FIG. 2 illustrates four cuvettes assemblies 110, each of which has four openings leading to four different chambers that provide for optical measurement of the fluid samples in the four chambers. The optical measurement is preferably a forward-scattering signal measurement caused by bacteria in the fluid sample. The cuvette assemblies 110 are described in more detail in U.S. Pat. No. 9,579,648, titled “Cuvette Assembly Having Chambers for Containing Samples to be Evaluated through Optical Measurement,” issued on Feb. 28, 2017 from an application filed on Dec. 5, 2014, which is commonly owned and is hereby incorporated by reference in its entirety. A brief description of the cuvette assembly 110 is provided below with reference to FIGS. 6-8. The cuvette assemblies 110 can be filled with fluid samples automatically or manually. As shown, the cuvette assemblies 110 are filled through the use of a pipette.

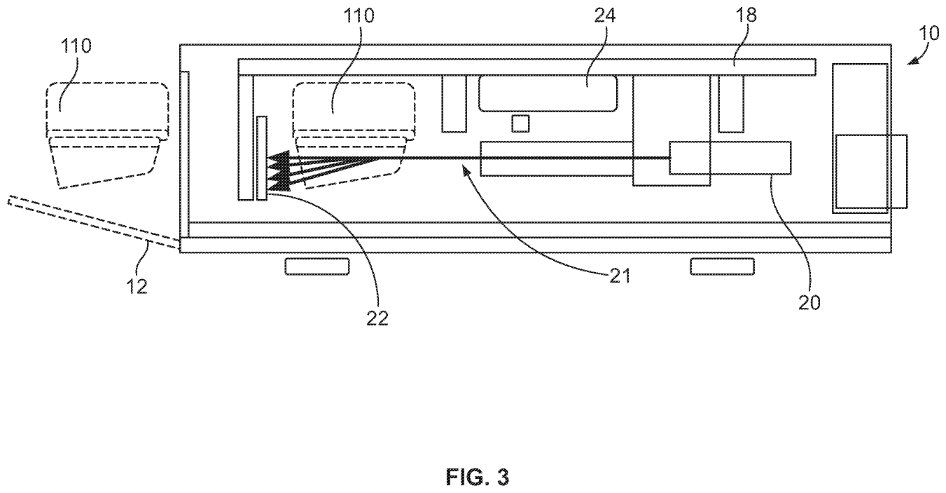

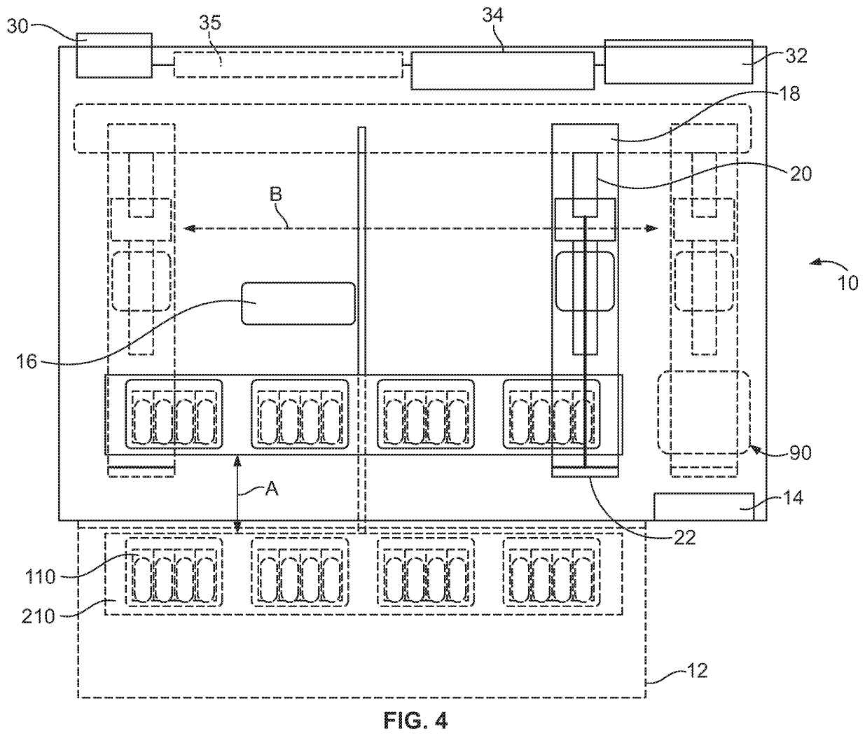

FIGS. 3-4 illustrate more of the details of the internal structures and components of the optical measurement instrument 10. In particular, as shown best in FIG. 4, the cuvettes assemblies 110 are loaded onto a movable platform 210 (show in detail in FIG. 8) when the door 12 is opened. Once loading is complete, the platform 210 moves inwardly into the instrument 10 and the door 12 is rotated to the closed position, creating a substantially light-tight seal. The door 12 has seals and/or gaskets around it so that the instrument 10 provides a light-tight enclosure to ensure proper signal detection by the sensor 22. As such, the movable platform 210 translates back and forth in the direction of arrow “A” in FIG. 4. The instrument 10 includes a motor 16, such as a motor that operates a gear (e.g., a worm gear) that is actuated to perform the platform movement and the opening and closing of the door 12.

An optical bench 18 is located within the instrument 10. A laser 20 (a light source), which provides an input beam 21, and a sensor 22 are coupled to the optical bench 18 in a fixed orientation. In one embodiment, the laser 20 is a visible wavelength collimated laser diode. In another embodiment the laser 20 is a laser beam delivered from an optical fiber. In yet another embodiment, the laser 20 includes multiple wavelength sources from collimated laser diodes that are combined into a single co-boresighted beam through one of several possible beam combining methods. In another example, the light source 20 is an incoherent narrow wavelength source such as an Argon gas incandescent lamp that is transmitted through one or more pinholes to provide a beam of directionality. A stepper motor 24 provides translation movement in the direction of arrow “B” to the optical bench 18, such that the laser 20 and the sensor 22 can move from side to side so as to be registered in 16 discrete positions that correspond to the 16 samples within the four cuvettes assemblies 110. At each position, the laser 20 is operational and its input beam 21 causes a forward-scatter signal associated with the liquid sample in question. The forward-scatter signal is detected by the sensor 22 and is associated with the bacteria concentration. As explained in more detail below with respect to cuvettes assemblies 110, each sample undergoes some type of filtering within the cuvette assembly 110 and/or outside the cuvette assembly 110 such that unwanted particles are substantially filtered, leaving only (or predominantly only) the bacteria. Due to the incubation feature within the instrument 10, the necessary environment around the cuvette assemblies 110 can be controlled to promote the growth of the bacteria, such that subsequent optical measurements taken by the combination of the laser 20 and the sensor 22 results in a stronger forward-scatter signal indicative of increased bacterial concentration. The instrument 10 includes internal programming that (i) controls the environment around the fluid sample and (ii) dictates the times and/or times-intervals between optical measurements to determine whether the bacteria has grown and, if so, how much the concentration of bacteria has increased. The output of the instrument 10 can be seen on a separate display.

In addition to the display 14 located on the instrument 10 (and preferably the input buttons and/or touchscreen on the instrument 10), the instrument 10 also includes a port 30 (e.g., a USB connection port) for communication with an external device such as a general purpose computer that would be coupled to the display. The instrument 10 can receive instructions from an external device that control the operation of the instrument 10. The instrument 10 can also transmit data (e.g., forward-scatter signal data, test-protocol data, cuvette-assembly data derived from a coded label 170 as shown in FIG. 6, diagnostic data, etc.) from the port 30. The instrument 10 also includes an input power port 32 (e.g., A/C power), which is then converted into a DC power supply 34 for use by the motors, laser, sensors, and displays, etc. One or more printed circuit boards 35 provide the various electronics, processors, and memory for operating the instrument 10.

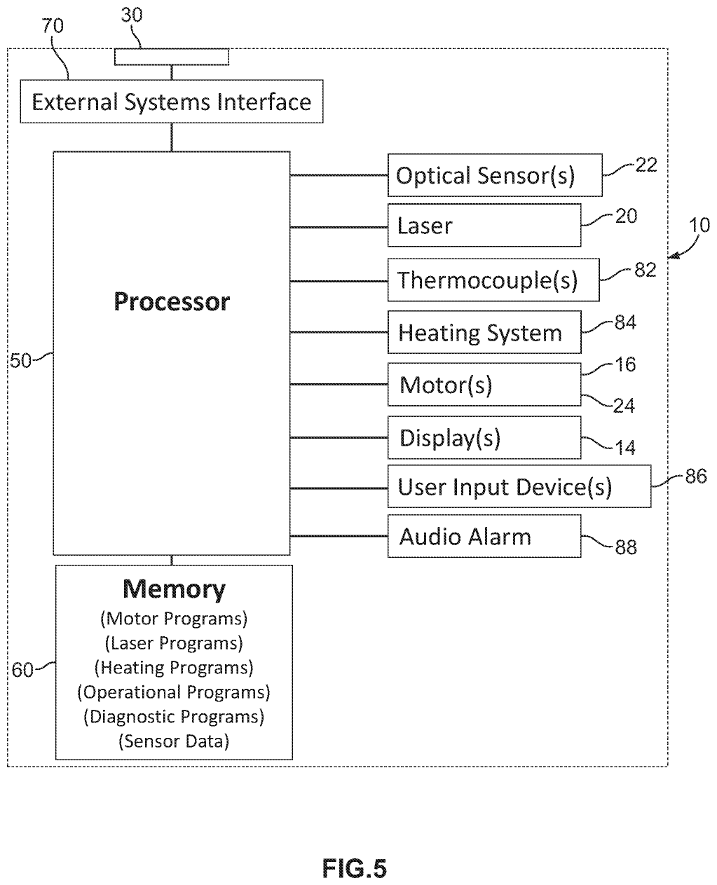

FIG. 5 illustrates one embodiment for a control system that is located within the instrument 10. The instrument 10 includes one or more printed circuit boards 35 that include at least one processor 50 (and possibly several processors) and at least one memory device 60. The processor 50 communicates with the memory device 60, which includes various programs to operate the motor(s), the laser, the sensors, the heating system, the basic operational functionality, diagnostics, etc. The processor 50 is in communication with the functional components of the instrument 10, such as (1) the optical sensor(s) 22 that sense the forward-scatter signals (or other optical signals, such as fluorescence signals), (2) the laser 20 or other light source that creates the light beam 21 is transmitted into the cuvettes, (3) thermocouple sensors 82 that determine the temperature within the enclosure (or associated with the surface of the cuvette, (4) the heating system 84, such as Kapton heaters, IR heaters, etc., which are preferably placed on the platform or tray 210 (FIG. 8) on which the cuvettes reside, (5) the motors 16, 24 used for opening the door, moving the platform, and moving the optical bench, (6) the display(s) 14 on the front of the instrument, (7) any user input devices 86 (mechanical buttons or touchscreens), and (8) an audio alarm 88 to alert the operator of the instrument to a particular condition or event (e.g., to indicate that one or more samples have reached a certain testing condition, such as a high bacterial concentration, a certain slope in a bacterial-growth curve has been achieved, or a certain forward-scatter signal exceeds a certain value).

The processor 50 is also communicating with an external systems interface 70, such as interface module, associated with the output port 30 on the instrument 10. The primary functions of the processor(s) 50 within the instrument 10 are (i) to maintain the enclosure within the instrument 10 at the appropriate temperature profile (temperature versus time) by use of the thermocouples 82 and heating system 84, (ii) to sequentially actuate the laser 10 so as to provide the necessary input beam 21 into the samples within the cuvette assemblies 110, (iii) to receive and store/transmit the data in the memory device 60 associated with the optical (e.g., forward-scatter) signals from the sensor(s) 22, and (iv) to analyze the forward-scatter signals to determine the bacterial concentration. Alternatively, the control system or computer module that controls the instrument 10 could be partially located outside the instrument 10. For example, a first processor may be located within the instrument 10 for operating the laser, motors, and heating system, while a second processor outside the instrument 10 handles the data processing/analysis for the forward-scatter signals received by the sensor 22 to determine bacterial concentration. The test results (e.g., bacterial concentration indication) and data from the instrument 10 can be reported on the instrument display 14 and/or transmitted by USB, Ethernet, Wi-Fi, Bluetooth, or other communication links from the external systems interface 70 within the instrument 10 to external systems that conduct further analysis, reporting, archiving, or aggregation with other data. Preferably, a central database receives test results and data from a plurality of remotely located instruments 10 such that the test data and results (anonymous data/results) can be used to determine trends using analytics, which can then be used to derive better and more robust operational programs for the instrument 10 (e.g., to decrease time per test, or decrease the energy of the tests by used lower incubation temperatures).

Referring to FIGS. 6-7, the cuvette assembly 110 includes four separate cuvettes, each of which includes an optical chamber 112 and a liquid-input chamber 114. The internal and external walls of the lower portion 113 of the main body of the cuvette assembly 110 define the optical chamber 112. For example, the first optical chamber 112 is partially defined by the side external wall, an internal wall, and a bottom wall of the lower portion 113, as well as the entry and exit windows 116, 118. The associated liquid-input chamber 114 is partially defined by a side external wall, an internal wall, and a pair of front and back external walls on the upper portion 115 of the main body of the cuvette assembly 110.

Each of the four entry windows 116 is a part of an entry window assembly 117 that is attached to the lower portion 113 of the main body of the cuvette assembly 110. Similarly, each of the four exit windows 118 is part of an exit window assembly 119 that is attached to the lower portion of the main body opposite the entry window assembly 117. In other words, the present invention contemplates a single unitary optical structure that provides the transmission of the input beam 21 into all four respective optical chambers 112, and a single unitary optical structure that provides for the exit of the forward-scatter signals from the respective optical chambers 112. The lower portion 113 of the main body includes structural recesses that mate with the corresponding structures on the window assemblies 117, 119 for registering them in a proper orientation during assembly of the cuvette assembly 110.

An intermediate partition 130 within the cuvette assembly 110 separates the lower portion 113 defining the four optical chambers 112 from the upper portion 115 defining the liquid-input chambers 114. The intermediate partition 130, which is shown as being part of the lower portion 113 (although it could be part of the upper portion 115), includes four separate groups of openings that permit the flow of liquid from the liquid-input chamber 114 into the associated optical chamber 112. The openings can be a variety of shapes that permit the flow of the liquid. As shown, the openings progressively get longer moving from the entry window 116 to the exit window 118 because the shape of the optical chamber 112 increases in area in the same direction. Additionally, the filter 132 rests upon the intermediate partition 130, such that the same filter 132 is used for each of the four regions. When the same filter 132 is used for all four regions, the interior walls of the upper portion 115 must provide adequate pressure at the filter 132 to prevent crossing fluid flows through the filter 132 between adjacent liquid-input chambers 112. In a further alternative, no filter 132 is present because the intermediate partition 130 includes adequate sized openings to provide the necessary filtering of the liquid sample, or because the liquid samples are pre-filtered before entering each liquid-input chamber 114.

To provide the initial introduction of the liquid samples into the cuvette assembly 110, the upper structure 138, which is attached to the upper portion 115 of the main body of the cuvette assembly 110, includes four openings 140 corresponding to the four liquid-input chambers 114. Four sliding mechanisms 142 are located within four corresponding grooves 144 on the upper structure 138 and are initially placed in an opened position such that the openings 140 are initially accessible to the user for introducing the liquid samples. Each of the sliding mechanisms 142 includes a pair of projections 148 that engage corresponding side channels at the edges of each of the corresponding grooves 144 to permit the sliding action. Within each groove 144, there is a latching ramp 146 over which the sliding mechanism 142 is moved when transitioning to its closed position. A corresponding latch 147 (FIG. 4) on the underside of the sliding mechanism 142 moves over the latching ramp 146 and creates a locking mechanism when the sliding mechanism 142 has been fully moved to the closed position. The upper structure 138 of the cuvette assembly 110 also includes a gripping handle 150 that permits the user to easily grasp the cuvette assembly 110 during transport to and from the platform 210 within the instrument 10 that incorporates the light source 20 and the sensor 22.

To help seal the cuvette assembly 110 after the liquid samples have been placed within the respective liquid-input chambers 114, the periphery of the sliding mechanism 142 adjacent to the opening 140 can be configured to tightly mate with the walls defining the groove 144 (or undercut channels within the groove 144) to inhibit any leakage around the opening 140 in the upper structure 138. Alternatively, a resilient plug-like structure can be located on the underside of the sliding mechanism 142 that fits within the opening 142 create a seal and inhibit leakage. Or, a gasket can be provided around the opening 140 to provide a sealing effect on the underside of the sliding mechanism 142. The cuvette assemblies 110 provide well sealed containment of the samples that reduces evaporation loss.

The upper portion 115 and the lower portion 113 of the main body of the cuvette assembly 110 can be attached to each other through various techniques, such as ultrasonic welding, thermal welding, with adhesive, or through interfering snap-fit connections. Similarly, the upper structure 138 can be attached to the upper portion 115 of the main body through similar techniques. And, the window assemblies 117, 119 can be attached to the lower portion 113 through the same attachment techniques. The width dimension of the overall cuvette assembly 110 across the four cuvettes is roughly 4 cm. The length dimension of the overall cuvette assembly 110 (i.e., parallel to the input beam) is approximately 2 cm. The height dimension of the overall cuvette assembly 110 is approximately 2 cm, such that each of the liquid input chambers 114 is approximately 1 cm in height and each of the optical chambers 112 is approximately 1 cm in height (although the optical chambers 112 have a varying height along the length direction due to their conical shape). In some embodiments, each optical chamber 112 is designed to contain approximately 1.2 to 1.5 cubic centimeters (i.e., approximately 1.2 to 1.5 milliliters) of a fluid sample. Each liquid-input chamber 114 is designed to hold slightly more of the liquid sample (e.g., 1.7 to 2.5 milliliters), which is then fed into the corresponding optical chamber 112.

Because each of the cuvette assemblies 110 may be used for different applications, the cuvette assembly 110 may use barcodes or RFID tags to identify the type of test supported by the particular cuvette assembly 110, as well as other measurement data to be taken. The instrument 10 that includes the light source 20 preferably reads the RFID or barcode, and selects the software program with the memory device 60 to run the appropriate optical measurement tests on the cuvette assembly 110. Accordingly, the cuvette assembly 110 preferably includes an identification label 170, which may include barcodes and/or quick response codes (“QR-code”) that provide the necessary coded information for the cuvette assembly 110. Other codes can be used as well. Specifically, when bacteria is a particle being checked within the liquid sample, one of the codes on the label 170 may provide the protocol for the test (e.g., temperature profile over duration of test, frequency of the optical measurements, duration of test, etc.), and the processor 50 executes instructions from the memory 60 (FIG. 5) corresponding to the test protocol. Another one of the codes may be associated with information on the patient(s) from whom the liquid samples were taken, which may include some level of encryption to ensure that patient data is kept confidential. Another code may provide a quality-assurance check of the part number or the serial number for the cuvette assembly 110 to ensure that the cuvette assembly 110 is an authentic and genuine part, such that improper cuvettes are not tested. The code for the quality-assurance check may also prevent a cuvette assembly 110 from being tested a second time (perhaps after some type of cleaning) if it is intended for only single use. Again, the instrument 110 preferably includes a device to read the codes associated with the label 170 (such as an image sensor, a barcode reader/sensor, or a QR-code reader/sensor). Alternatively, the codes on the label 170 can be scanned as the assemblies 110 are placed into the platform 210 (FIG. 8) such that the necessary information is obtained prior to the door 12 being closed.

The cuvette assembly 110 also includes a vent 180 (FIG. 7) that extends from the optical chamber 112 into the upper portion 115 of the main body the cuvette assembly 110. The vent 180 includes a chimney-like portion that extends upwardly from the intermediate partition 130. The chimney-like portion is then received in a channel in the upper portion 115, which extends to an opening 182 leading into the liquid-input chamber 114 just below the upper structure 138 that defines the upper boundary of the liquid-input chamber 114. Accordingly, the gas (e.g., air) that is initially present in the optical chamber 112 can be readily displaced as the optical chamber 112 receives the filtered liquid sample from the liquid-input chamber 114 (via the filter 132). The vent 180 can also lead to the external environment on the outside of the cuvette assembly 110.

FIG. 8 illustrates how the cuvettes assemblies 110 are registered with the optical measurement instrument 10 within a registration platform or tray 210, which is a part of the instrument 10. Each of the cuvettes assemblies 110 includes side registration features 192 that undergo a sliding engagement within corresponding vertical grooves 212 on pillars associated with the registration platform 210. Additionally, lower registration features 194 (FIG. 6) can slide within horizontal grooves 214 on an upper surface of the registration platform 210. The horizontal grooves 214 terminate in openings that receive the lower registration features 194 (illustrated as projections) on the cuvette assembly 110. Finally, the distance between the lower segments of the front and back walls of the cuvette assembly 110 corresponds to the width of the registration platform 210 such that cuvette assembly 110 becomes nestled between adjacent pillars with the front and back walls overlying the front and back edges of the registration platform 210.

As can be seen best in FIGS. 6-7, the lower surface of the lower portion 113 of the cuvette assembly 110, which includes the lower registration features 194, is at angle relative to the upper structure 138 of the cuvette assembly 110 and to the input beam from the light source 20 due to the conical geometry of the optical chamber 112. Accordingly, the upper surface of the registration platform 210 is angled in an opposing manner that allows the input beam to be generally horizontal (and generally parallel to the upper structure 138 of the cuvette assembly 110) when the cuvette assembly 110 is placed on the registration platform 210. It should be noted, however, that the cuvette assembly 110 can be properly registered on the registration platform 210 with less than these three distinct registration features illustrated in FIG. 8.

Once the cuvette assembly 110 is nestled properly on the registration platform 210, the door motor 16 is actuated, causing the now-loaded registration platform 210 to be pulled into the instrument 10 and the door 12 to be closed. The light source 20 can then sequentially transmit the input beam through each of the four optical chambers 112 of each cuvette assembly 110 and the forward-scatter signal associated with the particles within each of the liquid samples can be sequentially received by the sensor 22. The light source 20 and the sensor 22 on the optical bench 18 are controllably indexed between positions to receive optical measurements taken in adjacent optical chambers 112. As can be seen in FIG. 8, each platform 210 is capable of receiving four cuvette assemblies 110, such that optical measurements can be taken from sixteen different liquid samples within the four cuvette assemblies 110 nestled on the registration platform 210. Of course, the present invention contemplates an instrument 10 that uses more or less than four cuvettes assemblies 110.

According to this first embodiment, the instrument 10 has the optical beam 21 along a line from the laser 20 (or other light source such as an LED or lamp) and a light/image sensor 22 such as a camera, imager, calorimeter, thermopile, or solid-state detector array. The liquid samples are contained in the optical chambers 112 of the cuvette assemblies 110 between the light source 20 and the sensor 22 with at least one window so that light can transmit through the sample to the sensor 22. The light source 20 producing the optical beam 21 and the sensor 22 are rigidly mounted to a mechanical optical bench 18 (or plate), and the bench 18 is preferably mounted on rails or other mechanical structures for translational motion (or rotational motion) via a stepper motor 24 (or a motorized threaded stage that moves the bench, or a flexible motor-driven belt) so that it can be moved precisely relative to the sample in the cuvette 110 so that multiple samples can be optically measured. Additionally, the bench 18 could be translated to a diagnostic station 90 with no sample present (far right position of the optical bench 18 in FIG. 4) so that it can undergo self-testing or diagnostics in which the sensor 22 confirms performance of the light source 20, and the light source 20 confirms performance of the sensor 22, including provisions of a reticle or other optical devices that can be sensed to confirm alignment or optical power levels.

The sample-containing cuvettes 110 and the optical components are contained in an enclosure within the instrument 10 that excludes most ambient light, which might impact the measurement by the sensor. Alternatively, some portion of the sample cuvette or container could form a light-tight cover on the instrument, as described below in FIG. 9.

In this first embodiment, the sample-containing cuvettes 110 are disposable containers set on the platform 210 or tray or rail, which preferably includes the heating system 84, such as electrical resistance heaters or Peltier devices and the thermal sensors 82, such as common thermocouples. The heating system 84 and thermal sensors 82 form part of the incubation system that provide for appropriate temperature controls during operation of the instrument 10. The electronic control system in FIG. 5 provides for the thermostatic control of the temperature of the platform 210 and, thus, the contained liquid samples can be warmed or cooled (for example, through fans pulling in cooler air to the enclosure) to a set temperature to influence biological or chemical behavior of the liquid samples. Alternatively, the samples (and cuvettes 110) could be illuminated by optical or infrared (IR) light sources for heating, and the temperature can be measured or implied by direct or remote sensors.

Furthermore, the platform 210 may be equipped with a vibration-producing mechanism to help agitate the samples in the cuvettes 110. For example, a vibration motor can be coupled to the platform and 210 operated between cycles of the laser operation.

FIG. 9 illustrates an alternative optical-measuring instrument 310 that is capable of incubating fluid samples in cuvettes 312. However, unlike the previous embodiments, the cuvettes 312 form part of the light-tight closure of the optical-measuring instrument 310. In particular, the cuvettes 312 have an upper flange that rest on the exterior surface of the instrument 310. The exterior surface includes openings sized to receive the cuvettes in a certain notation, such that the upper flange rests against the exterior surface. When placed within the optical measuring instrument 310, the entrance and exit windows of the cuvettes are properly aligned with an input laser beam 321 from the laser 320 and the sensor 322 so as to provide proper registration for measuring the forward-scatter signal associated with the liquid sample. As in previous embodiments, the laser 320 and the sensor 322 would be mounted on an optical bench 318 that translates within the enclosure of the optical measuring instrument 310 by use of a stepper motor 324. As with the previous embodiments, the functions of the instrument 310 would be controlled by one or more processors 350. The optical bench 318 may include other optical components, such as lenses and apertures, to properly develop the laser beam 321 prior to transmission through the liquid sample in the cuvettes 312. The cuvettes 312 may have internal structures similar to those of the cuvette assemblies 110 in FIGS. 6-7.

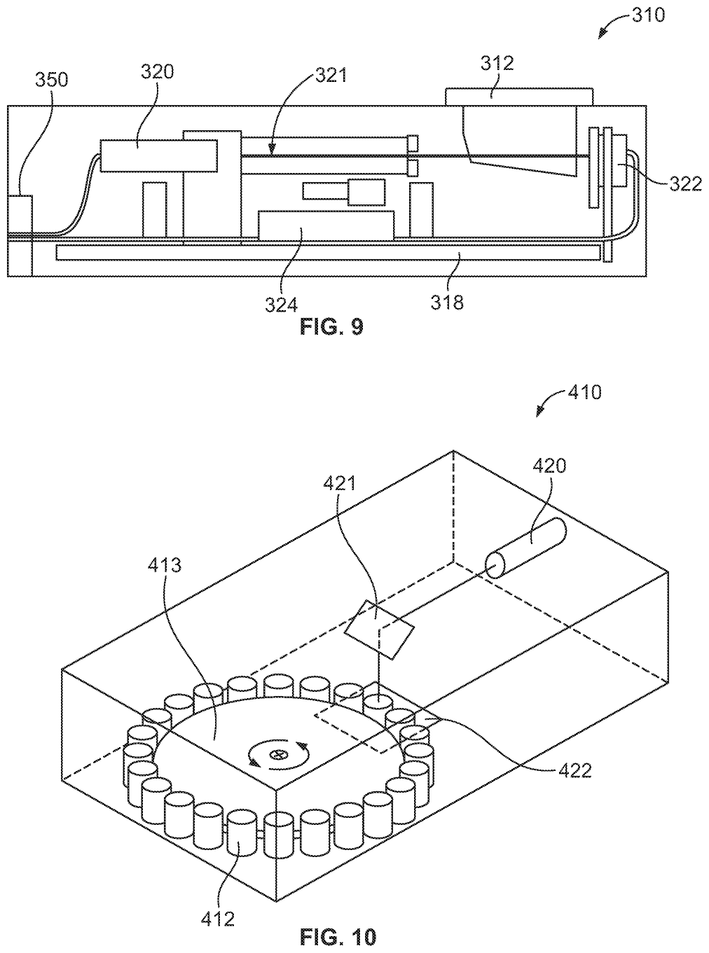

FIG. 10 illustrates another embodiment of an optical measuring instrument 410 that has one or more input beam lines that are fixed, which is different from the previous embodiments in which the beam lines are translated via the moving optical bench, which includes the laser and sensor. In the embodiment of FIG. 10, multiple sample chambers 412 (e.g., cuvettes) are held by a translatable or rotatable platform 413 that moves each sample into the light path within the optical measuring instrument 410. The light is developed by a light source, such as a laser 420 and may reflect off a turning mirror 421 before being transmitted through the fluid sample within the sample chamber 412. A sensor 422 receives the optical signal (e.g., a forward-scatter signal), which is then processed/analyzed to determine the presence and/or growth of bacteria over a period of time. The optical measuring instrument 410 may incorporate conductive heating and cooling, or radiant heating from an optical or infrared source for control of the temperature of the fluid samples, thereby providing the proper incubation.

In yet another embodiment of the instruments 10, 310, 410, the light source and sensor are fixed, and the multiple sample chambers are fixed. However, optical elements such as mirrors or prisms on electro-mechanical actuators are used to move the light beam from measurement chamber to measurement chamber within each sample. Hence, the electro-mechanical actuators and possibly motors are used to move the light beam, while the light source, the sensor(s), and the multiple sample chambers are fixed. In yet a further embodiment, there is a fixed sensor associated with each cuvette/sample position (e.g., such that the instrument has 16 individual sensors) and only the light source translates.

Regarding the operation of the instrument 10, one sample of test data from each fluid sample can be developed and recorded locally in the memory 60 within about 10 seconds. The laser 20 beam is transmitted through the sample contained between two windows, and into the sensor 22. The sensor 22 captures the scattered light across its surface and measures the distribution of light intensity as a forward scatter signal, which is them stored locally for a period of time, before being downloaded (on a periodic basis) to a larger memory device that is linked to the instrument 10. Similarly, the intensity of the laser beam on the sensor 22 can be measured in a location where there is no sample present, and again measured through the sample to determine the amount of power reduction that is attributable to absorption or reflectance of the enclosed sample, and the difference in these two values can be used to calculate optical density for the sample. As such, the instrument 10 can measure optical density of the fluid samples, which provides another piece of data that can be used for determining the bacterial concentration and its growth over a period of time. The optical bench 18 then translates to the position corresponding to the next sample. Accordingly, if sixteen samples are present (4 cuvette assemblies 110, each with 4 sample chambers), then the all sixteen samples can be completed in approximately 2-3 minutes. As such, the laser 20 and the sensor 22 continuously cycle through the fluid samples and measure a forward-scatter data point for each of the sixteen samples in about 2-3 minutes. For example, in a 2-hour test period, twenty or more multiple scatter signals for each of fluid samples can be taken.

The instrument 10 measures bacteria and other organisms generally in the range for 0.1 to 10 microns with a measurement repeatability of 10%. The instrument 10 can measure a low concentration of 1×10⁴ CFU/milliliters (based on E-coli in filtered saline) and deliver continuous measurements showing growth beyond 1×10⁹ CFU/milliliters. The instrument 10 can be loaded with factory-set calibration factors for approximate quantification of common organisms. Further, the user can load custom calibration factors with specific test protocols for use with less common organisms or processes.

Considering that the particles in the fluid (especially bacteria) may be in in motion, it is possible that large clusters may affect the forward-scatter signal on any given test sample. Accordingly, in one preferred embodiment, multiple consecutive test data points for each fluid sample are averaged to avoid having a single forward-scatter signal with a large cluster of particles or a single forward-scatter signal corresponding to only a few particles affect the overall test results. In one example, five consecutive forward-scatter signal test data points are averaged under a rolling-average method to develop a single average signal. Thus, as a new data point is taken for each sample, it is used with the previous four data points to develop a new average. More or less data points than five can be used for this rolling average. Further, the computation methodology may use various algorithms to remove the high and low signals (or certain ultra-high or ultra-low signals) before taking the average. Or, the computation methodology can be as simple as choosing the mathematical median of a data set. Ultimately, the forward-scatter signals from the instrument 10 will produce a bacterial-growth curve having a certain slope over a period of time at an appropriate incubation temperature.

Generally, growth curves are numerically filtered and analyzed for determination of initial concentration, growth percentage for a predefined period of time, and changes in the growth rate. Determination of bacterial absence or bacterial presence above a predefined threshold is based on a combination of those parameters with thresholds that are characteristic for bacterial growth and salts crystallization/dissolving kinetics. In one basic example, if the slope is above a predetermined value, the patient's sample is infected. Alternatively, it could be that the slope that indicates the presence of an infection may be different for different periods of time (e.g., Slope_{infection}>X within T=0 to 30 minutes; Slope_{infection}>1.5× within T=30 to 60 minutes; etc.)

Particles with a refractive index different from the surrounding medium will scatter light, and the resultant scattering intensity/angular distribution depends on the particle size, refractive index and shape. In situations in which the input light is scattered more than one time before exiting the sample (known as multiple scattering), the scattering also depends on the concentration of particles. Typically, bacteria have a refractive index close to that of water, indicating they are relatively transparent and scatter a small fraction of the incident beam, predominantly in the forward direction. With the optical design within the instrument 10, it is possible to look at scattering angles down to about 2° without having the incident input beam or other noise signals (e.g., the scattering from the cuvette windows) interfere with light scattered by bacteria. By simultaneous measurement of the forward scattering and optical density, measurements could be extended down to 10⁻⁵, allowing accurate measurement of concentrations as low as 10³ CFU/milliliters.

Optical density measurements are intended to determine sample concentrations that are not accurate, as the size of the scattering particles greatly affects the resulting optical density. A similar optical density is obtained for samples with a few large size bacteria in comparison with a higher concentration of small size bacterial samples. Moreover, additional calibration of the optical density to concentration does not render more accurate results, since the size changes during the bacterial growth process.

Bu use of the Mie scattering model for spherical particles and the T-matrix method of light scattering, combined with Monte-Carlo ray tracing calculation that takes into account multiple scattering, it is possible to evaluate the number of bacteria and their size from the measurement of the optical density and the scattered light angular distribution.

The results are nearly independent of the specific particle shape and loosely depend on the size dispersion of bacteria, resulting in a small constant shift of the mean size. Thus, both bacterial concentration and size are evaluated from the measured parameters by a first principle model without any free parameters, except the bacteria refractive index, that is measured by calibration for each of the bacteria species. In short, the instrument 10 can be used to detect forward scatter signals corresponding to scattering intensity and angular distribution (e.g., for angles less than 5°, such as angles down to about 2°) and also the optical density of the fluid samples, which can then be evaluated to determine the number of bacteria and their sizes (and changes to the number of bacteria and to their sizes over a period of time). The devices, systems, and methods described herein with respect to FIGS. 1-10 are generally similar to those described in U.S. Publication No. 2016-0160260, titled “Multi-Sample Laster-Scatter Measurement Instrument with Incubation Feature and Systems for Using the Same,” Dec. 4, 2015, which is commonly owned and is hereby incorporated by reference herein in its entirety.

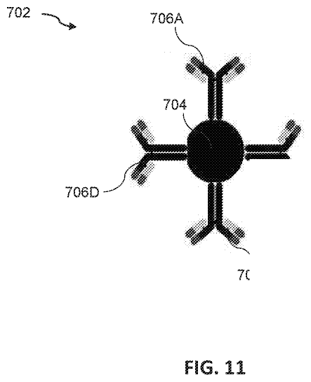

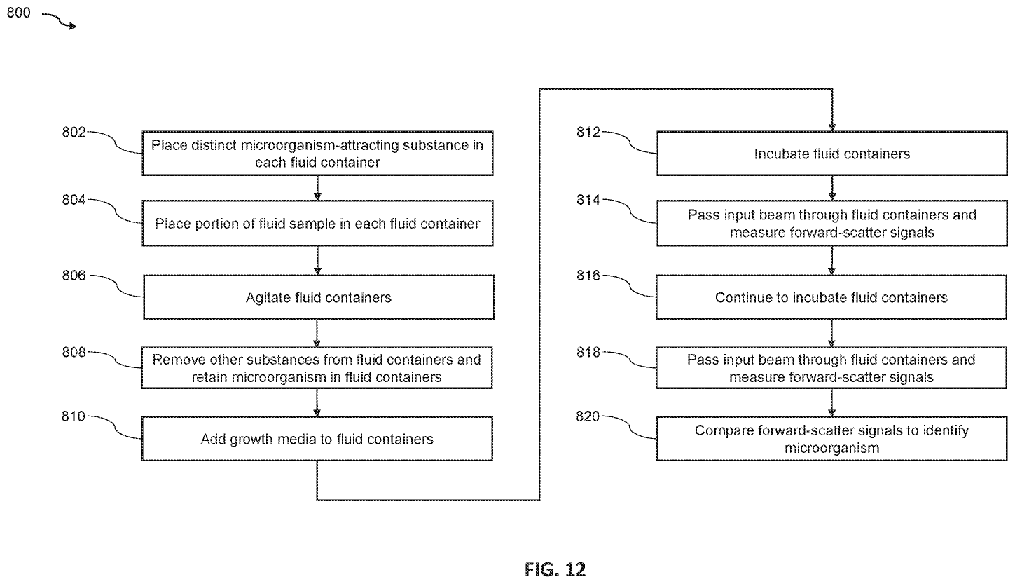

FIG. 11 shows a single molecule of a microorganism-attracting substance 702 that can be used to assist in detecting and identifying a microorganism (such as bacteria, yeast, fungi, etc.) in a fluid sample, using the devices, systems, and methods described herein. The microorganism-attracting substance 702 generally includes an affinity body 704 and one or more ligands 706A-706D coupled to the affinity body 704. The ligands 706A-706D are designed to bind generally with only a single genus, species, type, etc. of microorganism that may be present in the fluid sample. By using different types of ligands 706A-706D that bind with different types of microorganisms, multiple distinct microorganism-attracting substances 702 can be designed that attract and bind with only a single type of microorganism. By utilizing multiple different microorganism-attracting substances 702 in conjunction with the devices, systems, and methods described above, a user can simultaneously detect the presence of a microorganism within a fluid sample, and determine the identity of the microorganism.

In some implementations, the ligands 706A-706D are antibodies. Antibodies are generated by the body to target and defend against specific microorganisms. However, antibodies can also be artificially produced such that they bind only with a specific genus, species, type, etc. of microorganism, and do not bind with other genera, species, type, etc. of microorganism. These microorganism-specific antibodies can form a covalent bond with the affinity body 704 to form distinct microorganism-attracting substances 702. In other implementations, the ligands 706A-706D are microorganism-specific peptides. Similar to the antibodies, these peptides bind with the affinity body 704 and can be designed such that they strongly bind to only one type of microorganism. FIG. 11 shows four molecules of the ligand 706A-706D bonded to the affinity body 704. However, any suitable number of molecules of the ligand may be bonded to the affinity body 704.

The affinity body 704 is used as the base of the microorganism-attracting substance 702, and is configured to form a covalent bond with one or more molecules of the ligand 706A-706D. In some implementations, the affinity body 704 is made of a magnetic material. In other implementations, the affinity body 704 can be made of agarose. In some implementations, the affinity body 704 has a spherical shape, and can thus form a small bead. The spherical shape generally maximizes the amount of surface area on the affinity body 704 that is available to form covalent bonds with the ligands 706A-706D. In some implementations, the affinity body 704 can be manipulated by an external force to cause the affinity body 704 to move within a fluid container holding the microorganism-attracting substance 702. For example, when the affinity body 704 is made of magnetic material, a magnet external to the fluid container can be used to cause the affinity body 704 to move within the fluid container. The microorganism-attracting substance 702 is generally designed such that it does not interfere with the microorganism's ability to grow and reproduce when bound to the ligands 706A-706D of the microorganism-attracting substance 702.

A flowchart of a method 800 for detecting and identifying an unknown microorganism in a fluid sample is illustrated in FIG. 8. The fluid sample generally includes one or more molecules of the unknown microorganism, as well as one or more molecules of at least one other substance. For example, where the fluid sample is blood, the fluid sample may contain—in addition to the unknown microorganism—red blood cells, white blood cells, platelets, blood plasma, serum albumin, etc.

At step 802, a distinct microorganism-attracting substance is placed in each of a plurality of fluid chambers. The fluid chambers can be the same or similar to the optical chambers 112 in the cuvette assemblies 110, as discussed herein. Generally, each individual fluid container will contain a distinct microorganism-attracting substance that is configured to bind with only a single type of microorganism. A given fluid container is thus associated with the single distinct type of microorganism that is attracted to the distinct microorganism-attracting substance disposed in that fluid container. After filling all of the fluid containers with different microorganism-attracting substances, generally at least one of the fluid containers will be associated with the microorganism that is present in the fluid sample, which is still unknown at this point.

At step 804, a portion of the fluid sample is placed into each of the fluid containers. Because method 800 is generally used to detect and identify the unknown microorganism in a single fluid sample, each fluid container (associated with a single distinct type of microorganism) is be used to analyze the same fluid sample. In some implementations, different fluid containers could be used to analyze different fluid samples. If the microorganism-attracting substance within any of the fluid containers matches the unknown microorganism in the fluid sample, the unknown microorganism within that fluid container will generally begin to bind with the microorganism-attracting substance in that fluid container.

At step 806, the portions of the fluid sample are agitated to further facilitate interactions between the microorganism-attracting substance in each fluid container and the microorganisms within the fluid sample. In any fluid containers that include a microorganism-attracting substance that matches the unknown microorganism in the fluid sample, this agitation can increase the amount of molecules of the unknown microorganism that bind with the microorganism-attracting substance in the fluid container. The agitation of the fluid samples can be achieved via physical movement of the fluid containers, such as translational movement, rotational movement (e.g., movement about an internal axis), revolutionary movement (e.g., movement about an external axis), or any other suitable physical movement. The fluid samples can also be agitated by causing the affinity bodies to move within the fluid containers, for example via magnetic forces. Movement of the affinity bodies, and by extension the ligands bonded to the affinity bodies, can increase the likelihood that the affinity bodies and their associated ligands will encounter molecules of the unknown microorganism, thus increasing the amount molecules of the unknown microorganism that bind with the ligands. This allows a higher concentration of the unknown microorganism to be collected.

At step 808, substances other than the microorganism-attracting substance (and any unknown microorganisms bound to any of the microorganism-attracting substances) are removed from the fluid containers. For example, where the fluid sample is blood, substances such as red blood cells, white blood cells, plasma, etc. can be removed from the fluid containers. In the implementation where the affinity bodies are made of a magnetic material, magnets can be used to retain the affinity bodies, the ligands bonded to the affinity bodies, and any microorganisms bound to the ligands with the fluid sample. In any fluid container associated with a microorganism type other than the unknown microorganism in the fluid sample, the unknown microorganism will not be bound to the microorganism-attracting substance. The unknown microorganism will thus be removed from those fluid containers along, with the rest of the fluid sample. The microorganism-attracting substance will be retained in those fluid containers, but will not be bound to any of the unknown microorganisms from the fluid sample. Similarly, in any fluid container that is associated with unknown microorganism, the unknown microorganism will be bound to the microorganism-attracting substance, and thus will be retained within that fluid container. In an alternative implementation, the microorganism-attracting substances can be removed from each of the fluid containers and placed in different fluid containers. By removing the other substances (e.g. non-microbial substances) from the fluid containers, these other substances will not contribute to or interfere with the measurement system.

At step 810, an amount of a growth medium can be added to each of the fluid containers. The growth medium is generally clear such that light can be transmitted through the growth medium. The growth medium generally allows the bound microorganisms to grow and divide. In implementations where the microorganism-attracting substances and the unknown microorganism are removed from the initial fluid containers, they can subsequently be placed into other fluid containers that already have an amount of the growth medium disposed therein. In some implementations, the volume of the growth medium placed into the fluid containers with the microorganism-attracting substance (and at least one fluid container with the unknown microorganism) is less than the volume of the other substances within the fluid sample that were removed. This allows the same volume of the microorganism to be located within a smaller volume of other material, which increases the concentration of the unknown microorganism. This increased concentration can lead to reduced detection times.

At step 812, the fluid containers are incubated to encourage growth of the microorganism. At steps 814-820, the fluid containers can be analyzed using laser-scatter techniques as discussed herein. At step 714, an input beam (such as a laser beam) can be passed through each of the fluid containers, and thus through any of the substances within the fluid containers. The resulting first forward-scatter signal for each fluid container can be measured. At step 816, the fluid samples continue to be incubated for a period of time. At step 818, the input beam is again passed through each of the fluid containers and the resulting second forward-scatter signal for each fluid container can be measured.

At step 820, the differences between the first forward-scatter signal and the second forward-scatter signal for each fluid container can be measured. The forward-scatter signals are generally indicative of the growth of any microorganism within the fluid containers. Thus, by measuring the differences between the forward-scatter signals, it can be determined which fluid containers showed any microorganism growth. Because each fluid container is associated with a single type of microorganism, growth in any particular fluid container indicates that the type of microorganism associated with that particular fluid container was present in the original fluid sample. Thus, this determination of microorganism growth also determines the identity of the unknown microorganism in the fluid sample. The use of distinct microorganism-attracting substances therefore allows for simultaneous detection and identification of any unknown microorganisms within the fluid sample. In some implementations, a single type of unknown microorganism in the fluid sample is detected and identified. In other implementations, multiple types of unknown microorganisms in the fluid sample are detected and identified.

During steps 814-818, while the input beam is being passed through the fluid containers and the resulting forward-scatter signals are being measured, the affinity bodies can be manipulated (for example using a magnetic field) such that they are pulled out of the optical path of the input beam passing through the fluid containers. For example, the affinity bodies could be caused to sink to the bottom of the fluid containers. This ensures that the affinity bodies do not contribute to the forward-scatter signals or block the forward-scatter signal from reaching the sensor or other measurement equipment. While the sinking affinity bodies may carry any bound microorganisms out of the optical path, newly-grown microorganisms will remain in the solution in the optical path for detection.

While method 800 is described with reference to a first forward-scatter signal and a second forward-scatter signal, any number of forward-scatter signals can be generated and measured by the sensor to detect potential microorganism growth.

Further, while method 800 details how the unknown microorganism can be detected and identified using forward-scatter laser measurements, other types of measurement devices, systems, and methods are also contemplated. For example, the microorganism-attracting substance as disclosed herein can be used with optical density measurement, mass resonance, fluorescent markers, inherent fluorescence, cytometry, chemical detection of metabolic byproducts, or any other suitable types of measurement devices, systems, and methods.

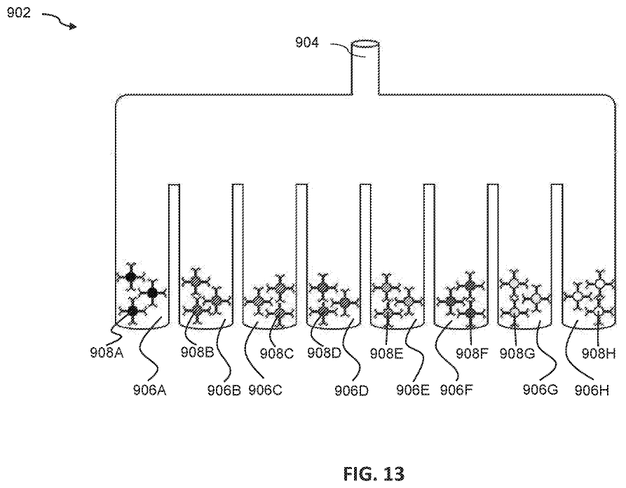

FIG. 13 shows a specimen collection device 902 that can be used collect portions of the fluid sample and hold the portions of the fluid sample during testing. The specimen collection device 902 generally includes an inlet 904 and a plurality of fluid containers 906A-906H. While specimen collection device 902 shows eight different fluid containers 906A-906H, any number of fluid containers could be used. Each of the fluid containers 906A-906H contains a respective distinct microorganism-attracting substance 908A-908H that is configured to attract a single genus, species, type, etc. of a microorganism. Each fluid container 906A-906H is thus associated with a single genus, species, type, etc. of a microorganism. In some implementations, specimen collection device 902 can be used as an initial collection device to obtain different portions of the fluid sample, agitate the fluid samples to assist in binding the microorganism-attracting substances with any corresponding microorganisms in the fluid sample, and separate out the other non-microbial substances in the fluid sample. The remaining microorganism-attracting substance (and potentially the unknown microorganism) can be transferred to another device or devices for the addition of the growth medium, incubation, and measurement. In other implementations, specimen collection device 902 can be used to hold the growth medium that is added, and serve as the fluid containers during incubation and measurement.

These embodiments and obvious variations thereof is contemplated as falling within the spirit and scope of the claimed invention, which is set forth in the following claims. Moreover, the present concepts expressly include any and all combinations and subcombinations of the preceding elements and aspects.

1. A specimen collection device, comprising:
an inlet; and
a plurality of fluid containers in fluid communication with the inlet, each of the plurality of fluid containers including a distinct microorganism-attracting substance disposed therein, the distinct microorganism-attracting substance of each of the plurality of fluid containers being configured to attract a respective one of a plurality of types of microorganisms such that each of the plurality of fluid containers is associated with the respective one of the plurality of types of microorganisms.

2. The specimen collection device of claim 1, wherein each of the microorganism-attracting substances includes an affinity body coupled to one or more molecules of respective one of a plurality of types of binding partners, the respective one of the plurality of types of binding partners of each distinct microorganism-attracting substance being configured to bind with the respective one of the plurality of types of microorganisms.

3. The specimen collection device of claim 2, wherein the affinity body of each distinct microorganism-attracting substances is comprised of a magnetic material or an agarose material.

4. The specimen collection device of claim 2, wherein the affinity body of each distinct microorganism-attracting substances has a generally spherical shape.

5. The specimen collection device of claim 2, wherein each of the plurality of types of binding partner includes a ligand.

6. The specimen collection device of claim 5, wherein the ligand includes an antibody, a peptide, or both an antibody and a peptide.

7. The specimen collection device of claim 1, wherein each of the plurality of types of microorganisms is attracted to the distinct microorganism-attracting substance within one of the plurality of fluid containers.

8. The specimen collection device of claim 1, wherein each distinct microorganism-attracting substance is configured to attract only the respective one of the plurality of types of microorganisms and not attract any other ones of the plurality of types of microorganisms.

9. The specimen collection device of claim 8, wherein each of the plurality of fluid containers is associated with only the respective one of the plurality of types of microorganisms and is not associated with any other ones of the plurality of types of microorganisms.

10. The specimen collection device of claim 1, wherein each of the plurality of fluid containers is sealed off the other fluid containers such that each of the plurality of fluid containers is in fluid communication with only the inlet.

11. An optical measuring instrument for detecting and identifying a microorganism in a fluid sample, comprising:
a housing with a substantially light-tight enclosure;
a plurality of fluid containers, each of the fluid containers holding a portion of the fluid sample and a distinct microorganism-attracting substance, the distinct microorganism-attracting substance of each of the plurality of fluid containers being configured to attract a respective one of a plurality of types of microorganisms such that each of the plurality of fluid containers is associated with the respective one of the plurality of types of microorganisms, each of the fluid containers having an input window and an output window;
a light source within the housing for providing an input beam for transmission into the input windows of the fluid containers and though the corresponding portions of the fluid sample, the input beam creating a forward-scatter signal for each of the fluid containers, each of the forward-scatter signals being associated with the presence and concentration of the respective one of the plurality of types of microorganisms associated with each of the plurality of fluid containers;
at least one sensor within the housing for detecting the forward-scatter signals exiting from the output windows; and
a heating element within the housing to maintain the portions of the fluid sample at a desired temperature to encourage microorganism growth in the portions of the fluid sample over a period of time,
wherein at least one of the input beam and the fluid containers are movable relative to each other so that the input beam sequentially addresses each of the plurality of fluid samples.

12. The optical measuring instrument of claim 11, wherein the movement of the input beam is effectuated by translational or rotational movement of an optical bench, and the plurality of fluid containers are held in a fixed position.

13. The optical measuring instrument of claim 11, wherein the heating element is part of an incubation system that further includes at least one temperature sensor for providing thermostatic control.

14. The optical measuring instrument of claim 11, wherein each of the microorganism-attracting substances includes an affinity body coupled to one or more molecules of respective one of a plurality of types of binding partners, the respective one of the plurality of types of binding partners of each distinct microorganism-attracting substance being configured to bind with the respective one of the plurality of types of microorganisms.

15. The optical measuring instrument of claim 11, wherein the light source and the sensor are fixed, and wherein the fluid containers are translated or rotated to move the portions of the fluid sample into the optical beam for sequential measurement of the forward-scatter signals.

16. The optical measuring instrument of claim 11, wherein the light source is a laser that is movable relative to the plurality of fluid containers, the laser periodically providing the input beam into each one of the portions of the fluid sample such that the forward-scatter symbols are detected over a period of time for each of the portions of the fluid sample.

17. An optical measuring instrument for detecting and identifying a microorganism in a fluid sample, comprising:
a plurality of cuvette assemblies having optical chambers for receiving a respective portion of the fluid sample, each of the optical chambers including (i) a distinct microorganism-attracting substance configured to attract a respective one of a plurality of types of microorganisms such that each of the plurality of optical chambers is associated with the respective one of the plurality of types of microorganisms, (ii) an entry window for allowing transmission of an input light beam through the respective portion of the fluid sample, and (iii) an exit window for transmitting an optical signal caused by the respective one of the plurality of types of microorganisms within the respective portion of the fluid sample, each cuvette assembly having a first pair of registration structures associated therewith;
a platform structure with multiple second pairs of registration structures for mating with the first pair of registration structures of the plurality of cuvette assemblies;
a light source producing the input light beam; and
a sensor for receiving the optical signal caused by the bacteria.

18. The optical measuring instrument of claim 17, wherein each cuvette assembly has the first pair of registration structures on a lower surface thereof, the lower surface being at an angle relative to a central axis of the input light beam.

19. The optical measuring instrument of claim 17, further including a moveable optical bench, the light source and the sensor being mounted on the optical bench, the movement permitting the portion of the fluid sample in each optical chamber to be individually measured.

20. The optical measuring instrument of claim 17, wherein each of the microorganism-attracting substances includes an affinity body coupled to one or more molecules of respective one of a plurality of types of binding partners, the respective one of the plurality of types of binding partners of each distinct microorganism-attracting substance being configured to bind with the respective one of the plurality of types of microorganisms.
